SEQUENCE LISTING (25 sequences)

SEQ ID NO: 1 - DNA, 633 bp, Pichia pastoris
atgagttttcaagtctacaacatggtgtccttgccagcaccgcaagcaacatcattctcccaacatccgaaacttccaacattggaagtgatacaacggccaccactcactggaagacatggcattcacgaagaagagatcaagctaccttcctttagatctttactggctagtgcgggcactgccgctgctcttgaatatccccccaggattcaaccccaatttgcaacaggggcacctgcacctgagcaagcattttatcagcccctaaataccgtgggagtcacgaggcccaacattcacacctccatcactccaccactggaggcgcaggacctccctcaccaacaaaagtttatggaaccatttgcgtccttccccaatacagtagtgtcttctacacccaacacagccatcagaaaatacaagtgcaagatctgcgagaggtctttcacaacgtcgggccacttagctcgtcacacacgtattcacactggtgaaaagaagcatgagtgtcctttcgaaggctgttctgctaggttcagccgtcaggataactgcatgcaacattacaagacgcatgttaataccaagtcgaaacgaaagagatctagattgagaagtaaataggta

SEQ ID NO: 2 - DNA, 327 bp, Pichia pastoris
atggctgaggaaaacagattatacgttaagggtaagcacgtgtctttccaacgttccaagagtgttatccacccaaagacttccttgatcaaaattgagggtgtcgaaaactcaaaggatgctgagttctacattggtaagagaatcgcttacgtctacaaaggtgtaaaggctatcaacggtacaaaggtcagagtcatgtggggaacagtcaccagaactcacggaaactctggtgttgtcagagctaagttcgaacgtaaccttccaggtcagagttttggttccactgtcagaatcatgctttacccatctaacatctaaatt

SEQ ID NO: 3 - DNA, 671 bp, Pichia pastoris
atggctgaggaaaacagatgtatgtactattaaatcgcaaggtgactggtaaatgatgaagatgatcggcttaatgagaagaataattgaaaacatagccagaaagttttgatttctgttaaaccaagctgaaatgatgggttgggaattttttattaagccaaatgaacattcttacaactttaaccaacggatattatatatggctgagccagcgttattccaaaagcacaagtgggaacaggtctctaagttttttctttactttcaagttgagttgaccgcttcatcttcattttctccgactatcaccaccttctcactatattttttagattcacgaggttactaactttgctatagtatacgttaagggtaagcacgtgtctttccaacgttccaagagtgttatccacccaaagacttccttgatcaaaattgagggtgtcgaaaactcaaaggatgctgagttctacattggtaagagaatcgcttacgtctacaaaggtgtaaaggctatcaacggtacaaaggtcagagtcatgtggggaacagtcaccagaactcacggaaactctggtgttgtcagagctaagttcgaacgtaaccttccaggtcagagttttggttccactgtcagaatcatgctttacccatctaacatctaaatt

SEQ ID NO: 4 - DNA, 621 bp, Pichia pastoris
atggctcccagaacactaccagaagacttaattccctccctatacgacttgcacatctacaacttccaacccgaaaaaaagacttatgatggagacattgtcatccacttggaggtgaaggagcccactgatgaagtggtcttcaatgccaaggatttggaattgaaagacgtacatgtcttccacaatgtcaacaagtctgaaaacgaaatccccgttaaggagattgttgataacgagctcatcacaattaagctcaaagagaaggttacttccggaacgttgctggtgaatatttccttcaccggtaacattcaatctgataaaattggattttacaagggagacacagatgtggaaggaagagtcacatacactacaaaccttaccactccaaatgccaggttggcattcccatgtcttgataacatattgttgaaagctccattcaagttcggagtaactgccaatccaggacaattagtgagttccattttggatctaagctctgaggctgacgtcttgaatgacaatgacgatgtgattggtacgagataccaataccaagtgagtgagccaatagccccagctttactggagtggaccattcatatttaacga

SEQ ID NO: 5 - DNA, 30 bp, Artificial Sequence
  primer 2F5 vH/hc FORW EcoRI
aggagaattcaggatcacgttaaaggaatc

SEQ ID NO: 6 - DNA, 26 bp, Artificial Sequence
  primer 2F5 vL/lc FORW EcoRI
acaagaattcgccctccaactgaccc

SEQ ID NO: 7 - DNA, 26 bp, Artificial Sequence
  primer cL BACK SacII
ccttccgcggctaacactctcccctg

SEQ ID NO: 8 - DNA, 32 bp, Artificial Sequence
  primer cH BACK EcoRI
gaattctttgtcacaagatttgggctcaactt

SEQ ID NO: 9 - DNA, 36 bp, Artificial Sequence
  primer Agg-EcorRI#1
aagaattcacagctagcgccaaaagctcttttatct

SEQ ID NO: 10 - DNA, 30 bp, Artificial Sequence
  primer Agg-XbaI#2
tgttctagattagaatagcaggtacgacaa

SEQ ID NO: 11 - DNA, 55 bp, Artificial Sequence
  oligonucleotide MCS#1
aattcttaattaagcagttaatgcggcgcgccatgcagcctatgcttaattaagc

SEQ ID NO: 12 - DNA, 55 bp, Artificial Sequence
  oligonucleotide MCS#2
ggccgcttaattaagcataggctgcatggcgcgccgcattaactgcttaattaag

SEQ ID NO: 13 - DNA, 36 bp, Artificial Sequence
  primer Pgap-back-EcoRI
gtcgtgaattcctcgtttcgaaatagttgttcaatt

SEQ ID NO: 14 - DNA, 41 bp, Artificial Sequence
  primer Pgap-middle-SfiI
aagagcattgctggccaagggggccgtagaagaagctctcc

SEQ ID NO: 15 - DNA, 32 bp, Artificial Sequence
  primer Pgap-for-BglII
tgcatgagatcagatcttttttgtagaaatgt

SEQ ID NO: 16 - DNA, 49 bp, Artificial Sequence
  primer GAPLibamp
accaccagaatcgaatataaaaggcgaacacctttcccaattttggttt

SEQ ID NO: 17 - DNA, 50 bp, Artificial Sequence
  primer Shble3out
gcgacccggccggcaactgcgtgcacttcgtggccgaggagcaggactga

SEQ ID NO: 18 - DNA, 49 bp, Artificial Sequence
  primer RPL33_#1_SbfI
gattcacctgcaggccatggctgaggaaaacagattatacgttaagggt

SEQ ID NO: 19 - DNA, 48 bp, Artificial Sequence
  primer RPL33_#2_SfiI
ggatggccgaggcggccttagatgttagatgggtaaagcatgattctg

SEQ ID NO: 20 - DNA, 53 bp, Artificial Sequence
  primer cLC52_#1_SbfI
gattcacctgcaggccatgagttttcaagtctacaacatggtgtccttgccag

SEQ ID NO: 21 - DNA, 54 bp, Artificial Sequence
  primer cLC52_#2_SfiI
ggatggccgaggcggccctatttacttctcaatctagatctctttcgtttcgac

SEQ ID NO: 22 - DNA, 55 bp, Artificial Sequence
  primer cLC61_#1_SbfI
gattcacctgcaggccatggctcccagaacactaccagaagacttaattccctcc

SEQ ID NO: 23 - DNA, 49 bp, Artificial Sequence
  primer cLC61_#2_SfiI
ggatggccgaggcggccttaaatatgaatggtccactccagtaaagctg

SEQ ID NO: 24 - DNA, 29 bp, Artificial Sequence
  primer KanR#1
ccgaggtaccgacatggaggcccagaata

SEQ ID NO: 25 - DNA, 30 bp, Artificial Sequence
  primer KanR#2
ccgaggtaccagtatagcgaccagcattca

(19) United States
(12) Patent Application Publication
(10) Pub. No.: US 2012/0064630 A1
(43) Pub. Date: Mar. 15, 2012

(54) EUKARYOTIC HOST CELL COMPRISING AN EXPRESSION ENHANCER

(75) Inventors: Gerhard Stadlmayr, Vienna (AT); Diethard Mattanovich, Vienna (AT); Michael Sauer, Vienna (AT)

(73) Assignee: FH CAMPUS WIEN, Vienna (AT)

(21) Appl. No.: 13/321,318

(22) PCT Filed: May 20, 2010
(86) PCT No.: PCT/EP2010/056967

§ 371 Date: Nov. 18, 2011

(30) Foreign Application Priority Data
May 20, 2009 (EP) 09160750.7

(51) Int. Cl. C12N 1/19; C12N 5/10; C12N 15/81; C12N 15/82; C12N 15/80; C12N 1/15; C12N 15/85

(52) U.S. Cl. 435/455; 435/254.23; 435/254.1; 435/325; 435/419; 435/468; 435/471; 435/320.1; 435/254.2

(57) ABSTRACT

Invention relates to a eukaryotic host cell comprising a recombinant nucleotide sequence encoding an expression enhancer, which is selected from the group consisting of cLC52, RPL33 and cLC61, and its use in a method of producing a protein of interest (POI).

The invention relates to a eukaryotic host cell comprising an expression enhancer and its use in a method to produce a protein of interest (POI).

Successful secretion of proteins has been accomplished both with prokaryotic and eukaryotic hosts. The most prominent examples are bacteria like Escherichia coli, yeasts like Saccharomyces cerevisiae, Pichia pastoris or Hansenula polymorpha, filamentous fungi like Aspergillus awamori or Trichoderma reesei, or mammalian cells like e.g. CHO cells. While the secretion of some proteins is readily achieved at high rates, many other proteins are only secreted at comparatively low levels (Punt et al., 2002; Macauley-Patrick et al., 2005; Porro et al., 2005).

One approach for the improvement of the secretion of a recombinant protein was done by random mutagenesis (Archer et al., 1994; Lang and Looman, 1995). The major disadvantage of this method is that positive results usually cannot be transferred to other strains.

The secretory pathway—the folding and processing of proteins—of eukaryotic organisms, e.g. of yeast, is very complex with many interacting participants. Some of these proteins have catalytic activity on the proteins like protein disulfide isomerase (PDI), others act by binding to the proteins and preventing them from aggregation (chaperones, e.g. BiP), or by stimulating release of the protein to the cell exterior at a later step in the secretory pathway (SSO proteins). Due to this interdependence, increasing the rate of one reaction step in the secretory pathway may not automatically augment secretion of a protein of interest, but instead may cause a rate-limitation at one or more of the subsequent reaction steps and thus may not remove but only shift bottle-neck(s) of the expression system.

It has been shown in several cases that the secretion process of heterologous proteins can be enhanced by co-overexpression of certain proteins which are called helper factors (Mattanovich et al., 2004).

WO 2008/128701A2 describes an expression system to increase the secretion of a POI from a eukaryotic cell employing a secretion helper factor selected from the group consisting of BMH2, BFR2, C0g6, C0Y1, CUPS, IMH 1, KIN2, SEC31, SSA4 and SSE1. The relevant genes were obtained from cDNA micoarrays of S. cerevisiae.

Gasser et al (Applied and Environmental Microbiology (2007) 6499-6507) describe the transcriptomics-based identification of novel factors enhancing heterologous protein secretion in yeasts. Specific secretion enhancers were found using DNA microarray hybridization experiments.

Co-expression of the gene encoding PDI and a gene encoding a heterologous disulphide-bonded protein was first suggested in WO 93/25676 as a means of increasing the production of the heterologous protein. WO 93/25676 reports that the recombinant expression of antistasin and tick anticoagulant protein can be increased by co-expression with PDI.

WO 94/08012 provides methods for increasing protein secretion in yeast by increasing expression of a Hsp70 chaperone protein, i.e. KAR2 and BiP or a PDI chaperone protein.

WO 03/057897 provides methods for the recombinant expression of a protein of interest by co-expressing at least two genes encoding proteins selected from the group consisting of the chaperone proteins GroEL, GRoES, Dnak, DnaJ, GRpe, CIpB and homologs thereof.

WO 2005/0617818 and WO 2006/067511 provide methods for producing a desired heterologous protein in yeast by using a 2 μm-based expression plasmid. It was demonstrated that the production of a heterologous protein is substantially increased when the genes for one or more chaperone protein(s) and a heterologous protein are co-expressed on the same plasmid.

Another approach to stimulate the secretory pathway is to overexpress the unfolded protein response (UPR) activating transcription factor HAC1. Transcriptional analyses revealed that up to 330 genes are regulated by HAC1, most of them belonging to the functional groups of secretion or the biogenesis of secretory organelles (e.g. ER-resident chaperones, foldases, components of the Translocon).

WO 01/72783 describes methods for increasing the amount of a heterologous protein secreted from a eukaryotic cell by inducing an elevated unfolded protein response (UPR), wherein the UPR is modulated by co-expression of a protein selected from the group consisting of HAC1, PTC2 and IRE1.

While these approaches, once established, can be transferred to other strains and used for other proteins as well, they are limited by the actual knowledge about the function of such proteins supporting the secretion of other proteins.

It can be anticipated that the successful high level secretion of a recombinant protein may be limited at a number of different steps, like folding, disulfide bridge formation, glycosylation, transport within the cell, or release from the cell. Many of these processes are still not fully understood. Such helper functions cannot be predicted with the current knowledge of the state-of-the-art, even when the DNA sequence of the entire genome of a host organism is available.

Wentz and Shusta (Appl. Environ. Microbiol. (2007) 73(4): 1189-1198) employ a Saccharamyces cerevisiae yeast surface display gene library to identify improved secretion strains using an appropriate selection pressure. The yeast cDNAs enhancing scTCR display were CCW12, SEDI, CWP2, RPPO, which were found to be temperature dependent, and ERO1 that was found to be a lone 20° C. enhancer.

It is an object of the invention to provide new methods to increase production of secreted proteins in eukaryotic cells, in particular in yeast cells of the Pichia genus, which are simple and efficient. It is desirable to provide new genes to be used in methods for the increased production of secreted proteins by overexpression of helper factors that could be employed in industrial production methods.

It is a further object of the present invention to provide a recombinant host cell with improved properties to increase production of a POI.

The object is solved by the subject matter as claimed.

In particular, the invention refers to a eukaryotic host cell comprising a recombinant nucleotide sequence encoding an expression enhancer, which is selected from the group consisting of cLC52, RPL33, cLC61, including functionally active variants thereof.

The host cell according to the invention preferably comprises the nucleotide sequence, which is originating from P. pastoris.

The host cell according to the invention preferably is a fungal cell, more preferably a yeast cell, or a higher eukaryotic cell, preferably a mammalian or a plant cell.

According to a specific aspect of the invention the host cell is a cell of the Pichia genus, in particular a cell of a strain of P. pastoris.

In one aspect the invention relates to such a host cell, which is a wildcard host cell for the introduction of a gene of interest encoding a POI.

In another aspect the invention relates to such a host cell, which is a producer cell comprising a recombinant nucleotide sequence encoding a POI, capable of producing the POI.

The invention preferably provides for a P. pastoris host cell comprising
a recombinant nucleotide sequence encoding an expression enhancer, which is selected from the group consisting of cLC52, RPL33 and cLC61, and
a gene of interest encoding a POI.

Such a nucleotide sequence encoding a protein selected from the group consisting of cLC52, RPL33 cLC61, including functionally active variants thereof, preferably is used according to the invention as an expression enhancer to increase the expression of a POI in a eukaryotic host cell.

The invention further provides for a method of producing a producer host cell, comprising
providing a wildcard host cell according to the invention that comprises the recombinant nucleotide sequence encoding a protein selected from the group consisting of cLC52, RPL33 cLC61, including functionally active variants thereof, and
introducing a nucleotide sequence encoding a POI.

According to an alternative embodiment, the method to produce a producer host cell comprises the steps of
providing a eukaryotic host cell,
introducing a nucleotide sequence encoding at least one of cLC52, RPL33, cLC61, including functionally active variants thereof, and
introducing a nucleotide sequence encoding a POI.

Thus, the invention provides for a method of producing a POI in a eukaryotic cell, comprising the steps of
providing a producer host cell according to the invention,
co-expressing the POI and the expression enhancer in a cell culture, and
obtaining the POI from the cell culture.

In a preferred method according to the invention the POI is a polypeptide, preferably a eukaryotic polypeptide, selected from the group consisting of serum proteins, such as an immunoglobulin or serum albumin, enzymes, hormones, signalling molecules, matrix proteins, fragments or derivatives thereof.

In another preferred method the POI is mediating the production of a metabolite of the host cell, preferably the POI is a substrate, an enzyme or a cofactor, and a metabolite is produced by the host to obtain a sufficient yield of the metabolite.

Preferably the recombinant nucleotide sequence encoding a POI is provided on a plasmid suitable for integration into the genome of the host cell or for autonomous replication in the host cell.

It is further preferred that the plasmid is a eukaryotic expression vector, preferably a yeast expression vector comprising a secretion leader sequence effective to cause secretion of the POI from the host cell and/or a promoter sequence effective to cause expression of the POI in the host cell.

In one aspect the invention relates to a co-overexpression plasmid for use in a method according to the invention comprising
the recombinant nucleotide sequence encoding a POI, and
a nucleotide sequence encoding a protein selected from the group consisting of cLC52, RPL33 cLC61, including functionally active variants thereof.

### DETAILED DESCRIPTION OF THE INVENTION

“Biologically active fragment” or “functionally active fragment” of a protein shall mean a fragment of a protein that exerts a biological effect similar or comparable to the full length protein. The functionally active fragment of the expression enhancer is characterized by being derived from the expression enhancers of SEQ ID NO: 1 to 4 by one or more deletions resulting in a polypeptide or the nucleotide sequence comprising at least 50% of the sequence, preferably at least 70%, more preferably at least 80%, still more preferably at least 90%, even more preferably at least 95% and most preferably at least 97%, 98% or 99%. Sequence identity may be determined as described below. Such fragments can be produced e.g. by amino- and carboxy-terminal deletion(s) as well as by internal deletion(s). Preferably the fragment is obtained by 1, 2, 3, 4, 5, 6, 7, 8, 9 or 10, more preferably 1, 2, 3, 4 or 5, even more preferably 1, 2 or 3, still more preferably 1 or 2, most preferably 1 deletion(s).

The term “derivative” encompasses any combination of one or more compounds, e.g. polypeptides or proteins, and/or a fusion compound, in which any part of the compound may be fused at any position to one or more other polypeptides. A derivative may also be obtained by association or binding to other substances by various chemical techniques such as covalent coupling, electrostatic interaction, di-sulphide bonding etc. The other substances bound to the compound may be lipids, carbohydrates, nucleic acids, organic and inorganic molecules or any combination thereof (e.g. PEG, prodrugs or drugs). The term “derivative” would also comprise a variant, also potentially containing non-natural or chemically modified amino acids, and fragments of compounds. It is understood that the terms cLC52, RPL33 or cLC61 also refer to derivatives thereof, which may be functional equivalents or functionally active variants, and/or have similar or improved functions.

“Expression vectors” as used herein are defined as DNA sequences that are required for the transcription of cloned recombinant nucleotide sequences, i.e. of recombinant genes and the translation of their mRNA in a suitable host organism. Such expression vectors usually comprise an origin for autonomous replication in the host cells, selectable markers (e.g. an amino acid synthesis gene or a gene conferring resistance to antibiotics such as zeocin, kanamycin, G418 or hygromycin), a number of restriction enzyme cleavage sites, a suitable promoter sequence and a transcription terminator, which components are operably linked together.

The term “eukaryotic host” shall mean any eukaryotic cell or organism, which may be cultivated to express a POI. It is well understood that the term does not include human beings.

The term “functionally equivalent variant” or “functionally active variant” of a polypeptide or a nucleotide sequence as used herein means a sequence resulting from modification of this sequence by insertion, deletion or substitution of one or more amino acids or nucleotides within the sequence or at either or both of the distal ends of the sequence, and which modification does not affect (in particular impair) the activity of this sequence. In a preferred embodiment the functionally active variant a) is a biologically active fragment of the polypeptide or the nucleotide sequence, the functionally active fragment comprising at least 50% of the sequence of the polypeptide or the nucleotide sequence, preferably at least 70%, more preferably at least 80%, still more preferably at least 90%, even more preferably at least 95% and most preferably at least 97%, 98% or 99%; b) is derived from the polypeptide or the nucleotide sequence by at least one amino acid substitution, addition and/or deletion, wherein the functionally active variant has a sequence identity to the polypeptide or the nucleotide sequence or to the functionally active fragment as defined in a) of at least 40%, preferably at least 60%, more preferably at least 75%, still more preferably at least 90%, even more preferably at least 95% and most preferably at least 97%, 98% or 99%; and/or c) consists of the polypeptide or the nucleotide sequence or a functionally active variant thereof and additionally at least one amino acid or nucleotide heterologous to the polypeptide or the nucleotide sequence, preferably wherein the functionally active variant is derived from or identical to any of the naturally occurring variants of any of the sequences of SEQ ID NO: 1, 2, 3 or 4.

Functionally active variants may be obtained by changing the sequence as defined above and are characterized by having a biological activity similar to that displayed by the respective sequence of SEQ ID NO: 1 to 4 from which the variant is derived, including the ability to enhance expression of a POI in a recombinant cell.

The functionally active variant may be obtained by sequence alterations in the polypeptide or the nucleotide sequence, wherein the sequence alterations retains a function of the unaltered polypeptide or the nucleotide sequence, when used in combination of the invention. Such sequence alterations can include, but are not limited to, (conservative) substitutions, additions, deletions, mutations and insertions.

The variant of the polypeptide or the nucleotide sequence is functionally active in the context of the present invention, if the activity of the composition of the invention including the variant (but not the original) amounts to at least 10%, preferably at least 25%, more preferably at least 50%, even more preferably at least 70%, still more preferably at least 80%, especially at least 90%, particularly at least 95%, most preferably at least 99% of the activity of the expression enhancer of the invention including the polypeptide or the nucleotide sequence without sequence alteration (i.e. the original polypeptide or the nucleotide sequence).

In one preferred embodiment of the invention, the functionally active variant of the peptide of the invention is essentially identical to the polypeptide or the nucleotide sequence of the SEQ ID NO: 1 to 4, but differs from the polypeptide or the nucleotide sequence, respectively, in that it is derived from a homologous sequence of a different strain or different species. These are referred to as naturally occurring variants.

However, the term “functionally active variant” includes naturally occurring allelic variants, as well as mutants or any other non-naturally occurring variants. As is known in the art, an allelic variant is an alternate form of a (poly)peptide that is characterized as having a substitution, deletion, or addition of one or more amino acids that does essentially not alter the biological function of the polypeptide. By “biological function” is meant a function of the polypeptide or the nucleotide sequence in the cell it naturally occurs in, even if the function is not necessary for the growth or survival of the cells.

In a preferred embodiment, the functionally active variant derived from the polypeptide or the nucleotide sequence as defined above by amino acid exchanges, deletions or insertions may also conserve, or more preferably improve, the activity.

Conservative substitutions are those that take place within a family of amino acids that are related in their side chains and chemical properties. Examples of such families are amino acids with basic side chains, with acidic side chains, with non-polar aliphatic side chains, with non-polar aromatic side chains, with uncharged polar side chains, with small side chains, with large side chains etc.

In another embodiment of the invention the polypeptide or the nucleotide sequence as defined above may be modified by a variety of chemical techniques to produce derivatives having essentially the same activity (as defined above for fragments and variants) as the modified polypeptide or the nucleotide sequence, and optionally having other desirable properties.

The term “gene product” or “product of a gene” is the biochemical material, either RNA or protein, resulting from expression of a gene, such as the cLC52, RPL33 or cLC61 genes.

As used herein, a “homologue” or “functional homologue” of a polypeptide shall mean that polypeptides have the same or conserved residues at a corresponding position in their primary, secondary or tertiary structure. The term also extends to two or more nucleotide sequences encoding homologous polypeptides. In particular, homologous compounds usually have at least about 50% amino acid sequence identity with regard to a full-length native sequence or any fragment thereof. Preferably, a homologous compound will have at least about 55% amino acid sequence identity, more preferably at least about 60% amino acid sequence identity, more preferably at least about 65% amino acid sequence identity, more preferably at least about 70% amino acid sequence identity, more preferably at least about 75% amino acid sequence identity, more preferably at least about 80% amino acid sequence identity, more preferably at least about 85% amino acid sequence identity, more preferably at least about 90% amino acid sequence identity, more preferably at least about 95% amino acid sequence identity to a native compound, or any other specifically defined fragment of a full-length compound. When the function as a helper factor is proven with such a homologue, the homologue is called “functional homologue”.

The term “homologous nucleotide sequences” as used herein refers to nucleotide sequences which are related but not identical in their nucleotide sequence with the contemplated nucleotide sequence, and perform essentially the same function. These are also meant to encompass variations in its nucleotide composition including variations due to the degeneracy of the genetic code, whereby the nucleotide sequence performs essentially the same function.

The term “host cell” or “hosts cell line” refers to a microorganism, used for expression of a recombinant gene to produce polypeptides or metabolites mediated by such polypeptides. A host cell clone of cultivated host cells that have proliferated is commonly understood to be a host cell line. A production host cell line is commonly understood to be a cell line ready-to-use for cultivation in a bioreactor to obtain the gene product in a production method.

The term “operably linked” as used herein refers to the association of nucleotide sequences on a single nucleic acid molecule, e.g. a vector, in a way such that the function of one or more nucleotide sequences is affected by at least one other nucleotide sequence present on said nucleic acid molecule. For example, a promoter is operably linked with a coding sequence of a recombinant gene, when it is capable of effecting the expression of that coding sequence.

“Percent (%) amino acid sequence identity” with respect to the polypeptide sequences identified herein is defined as the percentage of amino acid residues in a candidate sequence that are identical with the amino acid residues in the specific polypeptide sequence, after aligning the sequence and introducing gaps, if necessary, to achieve the maximum percent sequence identity, and not considering any conservative substitutions as part of the sequence identity. Those skilled in the art can determine appropriate parameters for measuring alignment, including any algorithms needed to achieve maximal alignment over the full length of the sequences being compared.

The terms “plasmid” and “vector” as used herein include autonomously replicating nucleotide sequences as well as genome integrating nucleotide sequences.

The term “polypeptide” refers to a protein or peptide that contains two or more amino acids, typically at least 3, preferably at least 20, more preferred at least 30, more preferred at least 50 amino acids. The term also refers to higher molecular weight polypeptides, such as proteins. Hereinafter the terms “polypeptide” and “protein” are interchangeably used.

“Promoter” as used herein refers to a DNA sequence capable of controlling the expression of a coding sequence or functional RNA. Promoter activity may be assessed by its transcriptional efficiency. This may be determined directly by measurement of the amount of mRNA transcription from the promoter, e.g. by Northern Blotting or indirectly by measurement of the amount of gene product expressed from the promoter.

The term “protein of interest (POI)” as used herein refers to a protein that is produced by means of recombinant technology in a host cell. More specifically, the protein may either be a polypeptide not naturally occurring in the host cell, i.e. a heterologous protein, or else may be native to the host cell, i.e. a homologous protein to the host cell, but is produced, for example, by transformation with a self replicating vector containing the nucleic acid sequence encoding the POI, or upon integration by recombinant techniques of one or more copies of the nucleic acid sequence encoding the POI into the genome of the host cell, or by recombinant modification of one or more regulatory sequences controlling the expression of the gene encoding the POI, e.g. of the promoter sequence.

The term “cLC52” shall mean a polypeptide of P. pastoris with homology to transcriptional regulators. In S. cerevisiae, the proteins with highest homology to cLC52 are Nrg1 and Nrg2, transcriptional repressors mediating glucose repression. Respective external IDs are: SGD YDR043C, SGD YBRO66C. Gene annotation PIPA03435.

The term “RPL33” shall mean a polypeptide of P. pastoris encoded by a rRNA gene with homology to S. cerevisiae RPL33A and RPL33B (Ribosomal protein L37 of the large (60S) ribosomal subunit). Respective external IDs are: SGD YPL143W and YDR234C. Gene annotation PIPA05837.

The term “cLC61” shall mean a polypeptide of P. pastoris encoded by a gene which is homologous to peptidases. Gene annotation PIPA02482.

As used herein the terms “cLC52, RPL33 or cLC61” are used interchangeably for the genes and the gene products. Sequence informations to these polypeptides are provided below. For the purpose of this invention, these factors are also called helper factors or expression enhancers. The terms shall encompass the full-length protein or functionally active variants thereof, including biologically active fragments, as well as functional homologues.

It was surprising to obtain such genes and gene products that could be used as protein expression and/or secretion, which have not been characterized as functional helper factor so far. It was found that in this regard relevant genes were cLC52, RPL33 and cLC61, which—upon co-expression could increase the yield in protein production. These genes have functions that would not assumably have been related to a POI expression and/or secretion.

To prove the helper function of the relevant genes, a cDNA library derived from different strains of P. pastoris, grown in a wide range of cultivation condition was co-overexpressed in a strain of P. pastoris displaying the Fab fragment of the monoclonal antibody on the cell surface. Strains with an increased fluorescence signal after immunfluorescence staining were enriched by using high throughput fluorescence activated cell sorting. After several rounds of culturing and sorting, preferably enriched in several rounds of sorting with increased stringency, e.g. in a very stringent single cell mode, the genes RPL33, cLC52 and cLC61 could be identified, acting positively on the amount of the surface-displayed Fab fragment, which have not been associated to the secretory pathway before. Such helper factors have not been identified before using the conventional DNA microarray technique.

Co-expression of one or more of the genes has a positive influence on the POI expression resulting in an increase of product concentration even with a reduction of biomass accumulation. Thereby a higher specific product yield may be obtained.

A more detailed description of the experimental procedure can be found in Example 1. The identified genes were amplified by PCR from P. pastoris cDNA and cloned into a P. pastoris expression vector and transformed in strains of P. pastoris pre-selected for high level production of various different POI, either surface-displayed or secreted. To estimate the effect of the co-overexpressed gene on the amount of surface-displayed or secreted recombinant POI, the obtained P. pastoris strains according to the invention may be cultured in shake flask experiments and fedbatch or chemostat fermentations in comparison with strains only expressing the POI. All three genes result in an increased cell surface signal in a co-overexpression study. In particular, co-overexpressed cLC52 significantly increased the amount of secreted POI.

According to preferred mode of the invention the recombinant nucleotide sequence comprises the cLC52 gene, which is Seq. ID No. 1, or functionally active variants thereof.

According to another preferred mode of the invention the recombinant nucleotide sequence comprises the RPL33 gene, which is Seq. ID No. 2 and 3, respectively, or functionally active variants thereof.

According to another preferred mode of the invention the recombinant nucleotide sequence comprises the cLC61 gene, which is Seq. ID No. 4, or functionally active variants thereof.

By means of the inventive helper factors the method according to the invention preferably not only refers to an increased production by an enhanced expression, but also to an increased secretion of the POI in a eukaryotic cell. An increase in secretion of the POI is determined on the basis of a comparison of its secretion yield in the presence or absence of co-expression of a said protein that increases protein secretion.

The POI can be any eukaryotic or prokaryotic polypeptide. It can be a naturally secreted protein or an intracellular protein, i.e. a protein which is not naturally secreted. The present invention also provides for the recombinant production of functional homologues, functionally active variants, derivatives and biologically active fragments of naturally secreted or not naturally secreted proteins. Functional homologues are preferably identical with or correspond to and have the functional characteristics of a sequence

A secreted POI referred to herein may be, but is not limited to, a protein suitable as a biopharmaceutical substance like an antibody or antibody fragment, growth factor, hormone, enzyme, vaccine, or a protein which can be used for industrial application like e.g. an enzyme.

The POI is preferably a heterologous recombinant polypeptide or protein, which may advantageously be produced in a eukaryotic cell, preferably a yeast cell, preferably as secreted proteins. Examples of preferably produced proteins are immunoglobulins, aprotinin, tissue factor pathway inhibitor or other protease inhibitors, and insulin or insulin precursors, insulin analogues, growth hormones, interleukins, tissue plasminogen activator, transforming growth factor a or b, glucagon, glucagon-like peptide 1 (GLP-1), glucagon-like peptide 2 (GLP-2), GRPP, Factor VII, Factor VIII, Factor XIII, platelet-derived growth factorl, serum albumin, enzymes, such as lipases or proteases, or a functional analogue of any one of these proteins. In the present context, the term “functional analogue” is meant to indicate a polypeptide with a similar function as the native protein. The polypeptide may be structurally similar to the native protein and may be derived from the native protein by addition of one or more amino acids to either or both the C- and N-terminal end or the side-chain of the native protein, substitution of one or more amino acids at one or a number of different sites in the native amino acid sequence, deletion of one or more amino acids at either or both ends of the native protein or at one or several sites in the amino acid sequence, or insertion of one or more amino acids at one or more sites in the native amino acid sequence. Such modifications are well known for several of the proteins mentioned above.

In another embodiment, the POI is a eukaryotic protein or a biologically active fragment thereof, preferably an immunoglobulin or an immunoglobulin fragment such as a Fc fragment or a Fab fragment. Most preferably, the POI is a Fab fragment of the monoclonal anti-HIV1 antibody 2F5.

A POI can also be selected from substrates, enzymes, inhibitors or cofactors that provide for biochemical reactions in the host cell, with the aim to obtain the product of said biochemical reaction or a cascade of several reactions (e.g. a metabolite of the host cell). Examplary products can be vitamins, such as riboflavin, organic acids, and alcohols, which can be obtained with increased yields following the expression of a POI according to the invention.

In general, the host cell, from which the proteins are secreted can be any eukaryotic cell suitable for recombinant expression of a POI. Examples of preferred yeast cells used as host cells according to the invention include but are not limited to the Saccharomyces genus (e.g. Saccharomyces cerevisiae), the Pichia genus (e.g. P. pastoris, or P. methanolica), the Komagataella genus (K. pastoris, K. pseudopastoris or K. phaffii), Hansenula polymorpha or Kluyveromyces lactis.

Newer literature divides and renames Pichia pastoris into Komagataella pastoris, Komagataella phaffii and Komagataella pseudopastoris (Kurtzman, 2005). Herein Pichia pastoris is used synonymously for all, Komagataella pastoris, Komagataella phaffii and Komagataella pseudopastoris.

The yeast producer organism preferably used according to the invention may be any suitable yeast organism which, on cultivation, produces large amounts of the heterologous protein or polypeptide in question. Preferred examples of suitable yeast organisms are strains selected from the yeast species Saccharomyces cerevisiae, Saccharomyces kluyveri, Schizosaccharomyces pombe, Saccharomyces uvarum, Kluyveromyces lactis, Hansenula polymorpha, Pichia pastoris, Pichia methanolica, Pichia kluyveri, Yarrowia lipolytica, Candida sp., Candida utilis, Candida cacaoi, Geotrichum sp., and Geotrichum fermentans.

The most preferred yeast host cells are derived from methylotrophic yeast, such as from Pichia or Komagataella, e.g. Pichia pastoris, or Komoagataella pastoris, or K. phaffii, or K. pseudopastoris. Examples of the host include yeasts such as P. pastoris. Examples of P. pastoris strains include CBS 704 (=NRRL Y-1603=DSMZ 70382), CBS 2612 (=NRRL Y-7556), CBS 7435 (=NRRL Y-11430), CBS 9173-9189, and DSMZ 70877 (German Collection of Microorganisms and Cell Cultures), examples of S. cerevisiae strains include W303, CEN.PK and the BY-series (EUROSCARF collection). All of the strains described above have been successfully used to produce transformants and express heterologous genes.

In general, the proteins of interest referred to herein may be produced by methods of recombinant expression well known to a person skilled in the art.

It is understood that the methods disclosed herein may further include cultivating said recombinant host cells under conditions permitting the expression of the POI. A secreted, recombinantly produced POI can then be isolated from the cell culture medium and further purified by techniques well known to a person skilled in the art.

The nucleotide sequences encoding the proteins that increase protein secretion can be obtained from a variety of sources. The origin of a recombinant helper nucleotide sequence (also called “helper gene”) according to the invention is preferably from a plant, insect, fungal or bacterial species, preferably from yeast, more preferably from a yeast strain different from the host yeast strain to obtain a recombinant strain. A preferred Pichia host cell according to the invention, such as a P. pastoris host cell, contains the heterologous helper factor genes, which are preferably derived from a S. cerevisiae strain or another P. pastoris strain, different from the production host. In another specific embodiment the host cell according to the invention comprises a recombinant nucleotide sequence with the helper factor genes originating from the same strain as the host cell.

According to a specific embodiment of the invention a helper gene preferably is obtained from a source cell of the same genus as the production host cell. For example, a nucleotide sequence encoding one of the expression helper factor according to the invention may be derived from yeast, such as a S.cerevisiae strain, and be used to co-express the helper factor with a POI in a yeast, such as a S.cerevisiae producer host cell line. A specifically preferred embodiment relates to a recombinant nucleotide sequence encoding a helper factor originating from P.pastoris for use in a method to co-express the helper factor with the POI in a P.pastoris producer host cell line. The homologous origin of the nucleotide sequence facilitates its incorporation into the host cell of the same genus, thus enabling stable production of a POI possibly with increased yields in industrial manufacturing processes. Also, functional equivalent nucleotide sequences from other suitable yeasts or other fungi or from other organisms such as vertebrates can be used.

In another preferred embodiment of the invention a helper factor nucleotide sequence is used that is isolated from Saccharomyces cerevisiae, particularly as an enhancer of the secretion of a POI in a eukaryotic cell, preferably in a yeast cell and most preferred in a cell of a strain of P. pastoris.

According to the invention it is also possible to provide a wildcard host cell according to the invention, which is comprising at least one of the helper factor genes, and which is ready to incorporate a gene of interest encoding a POI. The wildcard cell line is thus a preformed host cell line, which is characerized for its expression capacity. This follows an innovative “wildcard” strategy for the generation of producer cell lines (also called expression host cell line) for the production of biopharmaceuticals, e.g. using site-specific recombinase-mediated cassette exchange. Such a new host cell facilitates the cloning of a GOI, e.g. into predetermined genomic expression hot spots within days in order to get reproducible, highly efficient production cell-lines.

According to the invention it is preferred to provide a P. pastoris host comprising the helper factor gene operably linked to the nucleotide sequence codening for the POI.

According to a preferred embodiment the method of the invention employs a recombinant nucleotide sequence encoding the POI, which is provided on a plasmid suitable for integration into the genome of the host cell, in a single copy or in multiple copies per cell. The recombinant nucleotide sequence encoding the POI may also be provided on an autonomously replicating plasmid in a single copy or in multiple copies per cell.

Alternatively, the recombinant nucleotide sequence encoding the POI and the recombinant nucleotide sequence encoding a protein that increases protein secretion are present on the same plasmid in single copy or multiple copies per cell.

The preferred method according to the invention employs a plasmid, which is a eukaryotic expression vector, preferably a yeast expression vector. Expression vectors may include but are not limited to cloning vectors, modified cloning vectors and specifically designed plasmids. The preferred expression vector as used in the invention may be any expression vector suitable for expression of a recombinant gene in a host cell and is selected depending on the host organism. The recombinant expression vector may be any vector which is capable of replicating in or integrating into the genome of the host organisms, also called host vector, such as a yeast vector, which carries a DNA construct according to the invention. A preferred yeast expression vector is for expression in a yeast selected from the group consisting of methylotrophic yeasts represented by the genera Hansenula, Pichia, Candida and Torulopsis.

In the present invention, it is preferred to use a pPICZ, pGAPZ, pPIC9, pPICZalfa, pGAPZalfa, pPIC9K, pGAPHis or pPUZZLE-derived plasmids as the vector.

According to a preferred embodiment of the present invention, a recombinant helper gene, POI gene or recombinant host cell is obtained by ligating the relevant genes into a vector and constructing a single vector carrying the genes, or separate vectors to carry the helper factor genes and the POI genes, respectively. These genes can be stably integrated into the host cell genome by transforming the host cell using such vectors. The polypeptides encoded by the genes can be produced using the recombinant host cell line by culturing a transformant, thus obtained in an appropriate medium, isolating the expressed POI from the culture, and purifying it by a method appropriate for the expressed product, in particular to separate the POI from the co-expressed helper factor.

The relevant genes can be modified to be highly expressed in the host cell according to the invention. The genes can be modified by optimizing the gene sequence to correlate with those codons most frequently used in the host. For example, the gene sequence can be optimized according to the codons used with genes which are highly expressed in the host.

Several different approaches for the POI expression and secretion in the eukaryotic host cell are preferred. Proteins are expressed, processed and secreted by transforming the eukaryotic organism with an expression vector harbouring DNA encoding the desired protein and a signal peptide, preparing a culture of the transformed organism, growing the culture and recovering the protein from the culture medium. The signal peptide may be the desired protein's own signal peptide, a heterologous signal peptide or a hybrid of a native and a heterologous signal peptide. In the present context, the term “signal peptide” is understood to mean a presequence which is present as an N-terminal sequence on the precursor form of an extracellular protein expressed in yeast. The function of the signal peptide is to allow the heterologous protein to be secreted to enter the endoplasmatic reticulum. The signal peptide is normally cleaved off in the course of this process. The signal peptide may be heterologous or homologous to the host organism producing the protein.

In a preferred aspect the invention relates to such a method, wherein the expression vector comprises a secretion leader sequence effective to cause secretion of the POI from the host cell. The presence of such a secretion leader sequence in the expression vector is required when the POI intended for recombinant expression and secretion is a protein which is not naturally secreted and therefore lacks a natural secretion leader sequence, or its nucleotide sequence has been cloned without its natural secretion leader sequence. The secretion leader sequence may originate from yeast source, e.g. from yeast α-factor such as MFα of Saccharomyces cerevisiae, or yeast phosphatase, from mammalian or plant source, or others. The selection of the appropriate secretion leader sequence is apparent to a skilled person.

Alternatively, the secretion leader sequence can be fused to the nucleotide sequence encoding a POI intended for recombinant expression by conventional cloning techniques known to a skilled person. In preferred embodiments, the nucleotide sequence of the POI is fused to a secretion signal, i. e. a peptide that targets protein to the secretory pathway where the signal peptide is cleaved and the protein released in the medium, for example alfa-factor, PHO or AGA-2.

Appropriate expression vectors comprise regulatory sequences suitable for expression of DNA encoding a heterologous polypeptide or protein in a eukaryotic host cell. Examples of regulatory sequences include promoters, operators, and enhancers, ribosomal binding sites, and sequences that control transcription and translation initiation and termination. The regulatory sequences may be operably linked to the DNA sequence to be expressed. For example, a promoter sequence is said to be operably linked to a coding sequence, if the promotor controls the transcription of the coding sequence.

The promoter may be any DNA sequence which shows transcriptional activity in the host cell and may be derived from genes encoding proteins either homologous or heterologous to the host. The promoter is preferably derived from a gene encoding a protein homologous to the host cell.

In a further aspect the invention relates to such a method, wherein the expression vector comprises a promoter sequence effective to cause expression of the POI in the host cell. To allow expression of a recombinant nucleotide sequence in a host cell, the expression vector may provide the recombinant nucleotide sequence with a functional promoter adjacent to the 5′ end of the coding sequence. The transcription is thereby regulated and initiated by this promoter sequence.

Suitable promoter sequences for use with mammalian host cells may include but are not limited to promoters obtained from the genomes of viruses, heterologous mammalian promoters, e.g. the actin promoter or an immunoglobulin promoter, and heat shock protein promoters.

Further suitable promoter sequences for use with yeast host cells may include but are not limited to promoters obtained from genes that code for metabolic enzymes which are known to be present at high concentration in the cell, e.g. glycolytic enzymes like triosephosphate isomerase (TPI), phosphoglycerate kinase (PGK), glyceraldehyde-3-phosphate dehydrogenase (GAPDH), alcohol oxidase (AOX), lactase (LAC) and galactosidase (GAL).

Preferred examples of suitable promoters are the yeast promoters, which contains a DNA sequence that functions as a promoter for gene transcription in yeast cells. Preferred examples are S. cerevisiae Mal, TPI, CUP, ADH or PGK promoters, or the P. pastoris glucose-6-phosphate isomerase promoter (PPGI), the 3-phosphoglycerate kinase promoter (PPGK) or glycerol aldehyde phosphate dehydrogenase promoter PGAP, the alcohol oxidase promoter (PAOX), formaldehyde dehydrogenase promoter (PFLD), isocitrate lyase promoter(PICL), translation elongation factor promoter (PTEF), and the promoters of P. pastoris enolase 1 (PENO1), triose phosphate isomerase (PTPI), alpha-ketoisocaproate decarboxylase (PTHI), ribosomal subunit proteins (PRPS2, PRPS7, PRPS31, PRPL1), heat shock protein family members (PSSA1, PHSP90, PKAR2), 6-Phosphogluconate dehydrogenase (PGND1), phosphoglycerate mutase (PGPM1), transketolase (PTKL1), phosphatidylinositol synthase (PPIS1), ferro-O2-oxidoreductase (PFET3), high affinity iron permease (PFTR1), repressible alkaline phosphatise (PPHO8), N-myristoyl transferase (PNMT1), pheromone response transcription factor (PMCM1), ubiquitin (PUBI4), single-stranded DNA endonuclease (PRAD2) and the promoter of the major ADP/ATP carrier of the mitochondrial inner membrane (PPET9).

In a preferred expression system the promoter is an inducible or a constitutive promoter. The promoter can be an endogenous promoter or heterologous to the host cell.

The DNA sequence encoding the helper factor and/or the POI may also be operably connected to a suitable terminator sequence, for example AOX1 (alcohol oxidase) terminator, CYC1 (cytochrome c) terminator, TEF (translation elongation factor) terminator.

Expression vectors may comprise one or more phenotypic selectable markers, e.g. a gene encoding a protein that confers antibiotic resistance or that supplies an autotrophic requirement, and an origin of replication recognized by the intended host cell to ensure amplification within the host. Yeast vectors commonly contain an origin of replication from a yeast plasmid, an autonomously replicating sequence (ARS), or alternatively, a sequence used for integration into the host genome, a promoter region, sequences for polyadenylation, sequences for transcription termination, and a selectable marker.

The procedures used to ligate the DNA sequences, e.g. coding for the helper factor and/or the POI, the promoter and the terminator, respectively, and to insert them into suitable vectors containing the information necessary for integration or host replication, are well known to persons skilled in the art, e.g. described by J. Sambrook et al., “Molecular Cloning 2nd ed.”, Cold Spring Harbor Laboratory Press (1989).

It will be understood that the vector, which uses the helper factor gene and/or the POI as an integration target, may be constructed either by first preparing a DNA construct containing the entire DNA sequence coding for the helper factor and/or the POI and subsequently inserting this fragment into a suitable expression vector, or by sequentially inserting DNA fragments containing genetic information for the individual elements, such as the signal, leader or heterologous protein, followed by ligation.

Also multicloning vectors, which are vectors having a multicloning site, can be used according to the invention, wherein a desired heterologous gene can be incorporated at a multicloning site to provide an expression vector. Expression vectors are vectors carrying a heterologous gene and used for expression of a heterologous compound encoded by such gene. In expression vectors, the promoter is placed upstream of the gene of the heterologous compound and regulates the expression of the gene. In the case of multicloning vectors, because the gene of the heterologous compound is introduced at the multicloning site, the promoter is placed upstream of the multicloning site.

The DNA construct as provided to obtain a recombinant host cell according to the invention may be prepared synthetically by established standard methods, e.g. the phosphoramidite method. The DNA construct may also be of genomic or cDNA origin, for instance obtained by preparing a genomic or cDNA library and screening for DNA sequences coding for all or part of the polypeptide of the invention by hybridization using synthetic oligonucleotide probes in accordance with standard techniques (Sambrook et al., Molecular Cloning: A Laboratory Manual, Cold Spring Harbor, 1989). Finally, the DNA construct may be of mixed synthetic and genomic, mixed synthetic and cDNA or mixed genomic and cDNA origin prepared by annealing fragments of synthetic, genomic or cDNA origin, as appropriate, the fragments corresponding to various parts of the entire DNA construct, in accordance with standard techniques.

Transformants according to the present invention can be obtained by introducing such a vector DNA, e.g. plasmid DNA, into a host and selecting transformants which over-express the POI in the presence of a helper factor. Host cells are treated to enable them to incorporate foreign DNAs by methods conventionally used for transformation of eukaryotic cells, such as the electric pulse method, the protoplast method, the lithium acetate method, and modified methods thereof. P. pastoris is preferably transformed by electroporation.

In another preferred embodiment, the yeast expression vector is able to stably integrate in the yeast genome, e. g. by homologous recombination.

A transformant host cell according to the invention obtained by transforming the cell with the helper factor and/or the POI genes may preferably first be cultivated at conditions to grow efficiently to a large cell number without the burden of expressing a heterologous protein. When the cell line is prepared for the POI expression, cultivation techniques are chosen to produce the expression product.

When the transformant is grown with an inductive stimulus, a promoter may be activated to direct transcription of the gene under its control, and the POI is expressed. Under growth conditions with an inductive stimulus, it usually grows slower than under normal conditions, but because it has already grown to a large cell number in the previous stage, the culture system as a whole produces a large amount of the heterologous protein. An inductive stimulus is preferably heat, or addition of cadmium, copper, an osmotic pressure increasing agent, hydrogen peroxide, ethanol, methanol, methylamine or the like.

It is preferred to cultivate the host cell line according to the invention in a bioreactor under growth conditions to obtain a cell density of at least 1 g/L, more preferably at least 10 g/L cell dry weight, preferably at least 50 g/L cell dry weight. It is advantageous to provide for such yields of biomolecule production on a pilot or industrial scale.

The POI is preferably expressed employing conditions to produce yields of at least 1 mg/L, preferably at least 10 mg/L, preferably at least 100 mg/L, most preferred at least 1 g/L.

According to the present invention it is surprisingly possible to effectively co-express the helper factor and the POI with high yields, even when the biomass is kept low. Thus, a high specific yield, which is measured in mg POI/g dry biomass, in the range of 2 to 200, preferably 100 to 200, up to 500 in the pilot and industrial scale is thus feasible. The specific productivity of a production host according to the invention preferably provides for an increase of at least 1,1 fold, more preferably at least 1,5 fold, at least 1,2 or at least 1,3 fold, in some cases an increase of more than 2 fold can be shown, when compared to the expression of the product without the helper factors.

The host cell according to the invention is preferably tested for its expression capacity or yield by the following test: ELISA, activity assay, HPLC, or other suitable tests.

Preferred fermentation techniques are batch, fed batch or continuous cultivation.

Preferably the yeast is cultivated in a mineral medium with a suitable carbon source, avoiding a vitamin cocktail, thereby further simplifying the isolation process significantly. An example of a preferred mineral medium is one containing an utilizable carbon source (e.g. glucose, glycerol or methanol), salts containing the macro elements (potassium, magnesium, calcium, ammonium, chloride, sulphate, phosphate) and trace elements (copper, iodide, manganese, molybdate, cobalt, zinc, and iron salts, and boric acid).

The transformed cells are cultivated under conditions suitable to effect expression of the desired recombinant compound, which can be purified from the cells or culture medium, depending on the nature of the expression system and the expressed protein, e.g. whether the protein is fused to a signal peptide and whether the protein is soluble or membrane-bound. As will be understood by the skilled artisan, cultivation conditions will vary according to factors that include the type of host cell and particular expression vector employed.

If the desired compound is secreted from the cells, it can be isolated and purified from the culture medium using state of the art techniques. Secretion of the recombinant expression products from the yeast cells is generally advantageous for reasons that include facilitating the purification process, since the products are recovered from the culture supernatant rather than from the complex mixture of proteins that results when yeast cells are disrupted to release intracellular proteins.

Secreted proteins generally are initially expressed as precursors bearing an N-terminal signal or leader peptide. Signal peptides generally contain a positively charged N-terminus followed by a hydrophobic core, followed by a recognition site for an enzyme known as signal peptidase. This enzyme cleaves the signal peptide from the protein during translocation. The protein is transported from the endoplasmic reticulum to the Golgi apparatus, and then follows one of a number of routes in the secretory pathway, depending on the nature of the protein. The protein may be secreted into the culture medium or may be retained on the cell surface, for example. Certain receptors that comprise extracellular, transmembrane, and cytoplasmic domains are examples of proteins that may be retained on the cell membrane, with only the extracellular domain located outside the cell.

The cultured transformant cells may also be ruptured sonically or mechanically, enzymatically or chemically to obtain a cell extract containing the desired POI, from which the POI is isolated and purified.

As isolation and purification methods for obtaining a recombinant polypeptide or protein product, methods, such as methods utilizing difference in solubility, such as salting out and solvent precipitation, methods utilizing difference in molecular weight, such as ultrafiltration and gel electrophoresis, methods utilizing difference in electric charge, such as ion-exchange chromatography, methods utilizing specific affinity, such as affinity chromatography, methods utilizing difference in hydrophobicity, such as reverse phase high performance liquid chromatography, and methods utilizing difference in isoelectric point, such as isoelectric focusing may be used. Specific purification steps are preferably employed to separate any helper factor that is co-expressed and would contaminate the POI preparation.

The isolated and purified POI can be identified by conventional methods such as Western blotting or assay of its activity. The structure of the purified compound can be defined by amino acid analysis, amino-terminal analysis, primary structure analysis, and the like. It is preferred that the compound is obtainable in large amounts and in a high purity level, thus meeting the necessary requirements for being used as an active ingredient in pharmaceutical compositions.

The preferred host cell line according to the invention maintains the integration of the helper factor and POI genes, and the expression level remains high, e.g. at least at a pg level, even after about 20 generations of cultivation, preferably at least 30 generations, more preferably at least 40 generations, most preferred of at least 50 generations. The recombinant host cell is surprisingly stable, which is a great advantage when used for industrial scale protein production.

The present invention is described in further detail in the following examples, which are not in any way intended to limit the scope of the invention as claimed.

### EXAMPLES

### Example 1

a) Construction of P. pastoris Strain Displaying Fab 2F5 on the Surface

For the generation of the Fab 2F5 expression cassettes, the entire light chain genes (vL and cL) and the vH and cH1 region of the heavy chain genes were amplified by PCR from pRC/RSV containing the humanized IgG1 mAb (Kunert et al., 1998, 2000). Non template codes restriction sites were added by using oligonucleotide primer (Table 1)

TABLE 1
Oligonucleotide primer for PCR amplification of
Fab 2F5 heavy and light chain

| | |
|---|---|
| 2F5 vH/hc | 5′-AGGAGAATTCAGGATCACGTTAAaGGAATC-3′ |
| FORW EcoRI | (SEQ ID No. 5) |
| 2F5 vL/Ic | 5′-ACAAGAATTCGCCCTCCAACTGACCC-3′ |
| FORW EcoRI | (SEQ ID No. 6) |
| cL BACK | 5′-CCTTCCGCGGCTAACACTCTCCCCTG-3′ |
| SacII | (SEQ ID No. 7) |
| cH BACK | 5′-GAATTCTTTGTCACAAGATTTGGGCTCAACTT-3′ |
| EcoRI | (SEQ ID No. 8) |

The light chain fragments were ligated into the EcoRI and SacII site of a modified version of pGAPalfaA (Invitrogen), where the AvrII restriction site was changed to a NdeI site by site directed mutagenesis to allow subsequent linearization of the plasmids containing two expression cassettes.

The heavy chain fragments were inserted into the EcoRI site of a modified version of pGAPZalfaA carrying alfa-agglutinin-fragment (alfa-Agglutinin fragment was amplified by PCR from S.cerevisiae genomic DNA using Primer Agg-EcorRI #1 (SEQ ID No. 9) and Agg-Xbal #2 (SEQ ID No. 10) (Table 2) and cloned into the EcoRI and Xbal site of pGAPZalfaA).

TABLE 2
Oligonucleotide primer for PCR amplification of
alfa-Agglutinin for S. cerevisiae

| | |
|---|---|
| Agg- | 5′-AAGAATTCACAGCTAGCGCCAAAAGCTCTTTTATCT-3′ |
| EcorRI | (SEQ ID No. 9) |
| #1 | |
| Agg- | 5′-TGTTCTAGATTAGAATAGCAGGTACGACAA-3′ |
| XbaI#2 | (SEQ ID No. 10) |

A plasmid combining the expression cassettes for both Fab chains on one vector was produced by double digestion of the light chain vector with BgIII and BamHI, and subsequent insertion of this expression cassette into the unique BgIII site of the respective vector (pGAPZaA) already containing a single copy of the expression cassette of the heavy chain fragment fused to the alfa-agglutinin fragment.

To generate a plasmid conferring Geneticin (G418) resistance in P. pastoris, the Sh ble gene (conferring resistance to Zeocin (Zeocin™ is a trademark of CAYLA) of the plasmid pGAPZalfaA carrying the Fab 2F5 light chain and the heavy chain alfa-agglutinin fusion(pGAPZalfaA-LC-HCaAggl) was replaced by the Tn903 kanR gene from the kanMX4 cassette of pFA6KanMX4 (Longtine et.al 1998). The exchange was done by cloning the Tn903 kanR gene (pFA6kanMX4 cut with NcoI and ScaI) into the plasmid backbone of the pGAPZalfaA-LC-HCalfaAggl digested with NcoI and EcoRV.

The resulting plasmid was linearized with AvrII prior to electrotransformation into P.pastoris SMD1168 (Invitrogen).

To assure the display of the antibody fragment Fab 2F5 on the surface of P. pastoris flow cytometry analysis of immunofluorescent stained cells (with anti-human kappa light chain FITC conjugate; Sigma-Aldrich F3761) were performed and high producing (displaying) strains were selected.

b) Construction of the Plasmid pLIB Suitable for Co-Overexpression of a P. pastoris cDNA Library:

To generate pLIB in a first step the multiple cloning site (MCS) of pGAPZB (Invitrogen) was modified by inserting of an artificial oligonucleotide (annealed 5′phosphorelated oligonucleotide MCS #1 (SEQ ID No. 11) and MCS#2 (SEQ ID No. 12), (Table 3) coding for additional restriction sites BstAPI(A), PacI and BstAPI(B) between the EcoRI and NotI site of pGAPZB, The restriction sites BstAPI(A) and BstAPI(B) were designed to form compatible sticky-ends with Sfil(A)/Sfil(B). In a second step the original GAP promoter was replaced by a PCR fragment of a modified GAP promoter, carrying two additional by at position 238 (modified by PCR using the megaprimer method; oligonuceotide primer see Table 4) by cloning it between BgIII and EcoRI. The insertion of these two by led to a novel SfiI restriction site.

TABLE 3
Oligonucleotides for multiple cloning site

| MCS | 5′-PHO-AATTCTTAATTAAGCAGTTAATGCGGCGCGCCATGC |
|---|---|
| #1 | AGCCTATGCTTAATTAAGC-3′ (SEQ ID No. 11) |
| MCS | 5′-PHO-GGCCGCTTAATTAAGCATAGGCTGCATGGCGCGCCG |
| #2 | CATTAACTGCTTAATTAAG-3′ (SEQ ID No. 12) |

TABLE 4
Oligonucleotide primer for PCR amplifi-
cation of α-Agglutinin for S. cerevisiae

| | |
|---|---|
| Pgap- | 5′-GTCGTGAATTCCTCGTTTCGAAATAGTTGTTCAATT-3′ |
| back- | (SEQ ID No. 13) |
| EcoRI | |
| Pgap- | 5′-AAGAGCATTGCTGGCCAAGGGGGCCGTAGAAGAAGCTC |
| middle- | TCC-3′ (SEQ ID No. 14) |
| SfiI | |
| Pgap- | 5′-TGCATGAGATCAGATCTTTTTTGTAGAAATGT-3′ |
| for- | (SEQ ID No. 15) |
| BgIII | |

In another step the AOX1 transcription terminator and the Zeocin resistance cassette of the resulting plasmid pGAP(Sfil)MCSmod was changed against the CYC1 transcription terminator of S.cerevisiae—Zeocin resistance cassette fragment from the plasmid pPuzzle_ZeoR_Pgap_eGFP_AOXTT (Gasser et.al. 2008) To avoid gene replacement events (knockout of the expression cassettes of 2F5Fab heavy and/or 2F5Fab light chain) via homologous recombination the direction of the Zeocin resistence cassette was changed by restriction enzyme digest with Kpnl and re-ligation.

pPuzzle_ZeoR_Pgap_eGFP_AOXTT(reverse) was digested with BamHI (followed by treatment with Klenow-polymerase), and BssSI. The DNA fragment of the correct size was cloned into the plasmid pGAP(Sfil)MCSmod (digested with Agel, treated with Klenow and digested with BssSI). The resulting plasmid was called pLIB.

c) Construction of cDNA Co-Expression Library:

cDNA synthesis was performed starting from polyA-RNA pool of different strains of P. pastoris grown under different environmental conditions (modified cultivation parameter: temperature, pH, different carbon and nitrogen sources; complex and minimal-salt media, heat shock) following the protocol of the SMART Creator cDNA Library Construction Kit (Clontech) until the step of cloning the cDNA PCR fragments. Instead of cloning the cDNA into the provided plasmid, the resulting cDNA-PCR fragments were cloned into the plasmid pLIB (example 1 step b), which was especially designed and constructed for this study (cDNA library was amplified by PCR and digested with SfII and cloned into the BstAPI and alkaline phosphatase treated pLIB). Transformation of E. coli via electrotransformation led to 1×10⁶ individual clones. The efficiency of the cloning procedure was checked by restriction enzyme analysis.

The resulting plasmid library (linearized with Sfil for integration into the genome via homologous recombination) was used to transform a pre-selected strain of P. pastoris displaying the antibody fragment Fab2F5 on the surface of the cell (example 1 step a). An optimized transformation procedure led to a P. pastoris cDNA co-expression library with 1.2×10⁶ individual clones on YPD agar containing Zeocin. All colonies were scraped off from the plates using YPD media. The resulting cell suspension was used to inoculate an expression culture with a starting optical density at 600 nm of 0.1 (conditions: 100 ml shake flask culture with 10 ml BM media (per litre: 10 g yeast extract, 10 g peptone, 100 mM potassium phosphate buffer pH 6.0, 13.4 g yeast nitrogen base with ammonium sulfate, 0.4 mg biotin). Cells were grown at 28° C. and 180 rpm to mid-log phase (OD600 8-15). About 10⁷ cell were immunofluorescent stained with anti-human-kappa-light-chain FITC conjugate (Sigma-Aldrich F3761, dilution 1:100) in PBS containing BSA and after 3 seconds of sonication they were used for fluorescent activated cell sorting (FACS). FACS was performed on a FACS Calibur (Becton Dickinson, Franklin Lakes, N.J., USA). For FACS two gates had to be defined. Gate 1 was defined on the dotplot FSC vs. SSC and it was used to distinguish cells and cells debris. Gate 2 was designed on a dotplot FL1 vs. FSC in that way that it collects only those cells of gate 1 with the highest FL1 signal. Only events (cells) belonging to both gates were sorted and collected in a cells concentrator. The ratio of sorted events vs. unsorted events (Table 5) was controlled via moving gate 2. The recovery of the sorted cells was done in liquid BM media. All recovered cells were used to inoculate a fresh expression culture and grown under above described conditions to mid-log phase again.

This procedure was repeated four times, so that a total of five consecutive rounds of sorting were performed.

TABLE 5
Sorting rounds

| sorting round | total events | sorted events | | Sorting mode |
|---|---|---|---|---|
| 1 | 1.007 × 10⁷ | 2.59 × 10⁵ | 2.59% | exclusion mode |
| 2 | 1.02 × 10⁷ | 1.46 × 10⁵ | 1.46% | exclusion mode |
| 3 | 1.50 × 10⁷ | 3.12 × 10⁴ | 0.207% | exclusion mode |
| 4 | 1.84 × 10⁷ | 1.76 × 10⁴ | 0.095% | exclusion mode |
| 5 | 2.10 × 10⁷ | 1.01 × 10⁴ | 0.048% | single cell |

After the 5^{th} round of expression, staining and sorting, all sorted cells were plated on YPD agar (per litre: 10 g yeast extract, 20 g peptone, 20 g glucose, 25 mg Zeocin, 500 mg G418-sulphate). After one round of replica plating, to individualize single clones, these single colonies were used to inoculate expression cultures and grown to mid-log phase again. An aliquot of cells from each individual clone was used for immunofluorescence staining followed by flow cytometry analysis. Flow cytometric analyses were performed on a FACS Calibur (Becton Dickinson, Franklin Lakes, N.J., USA). The probe was excited with a 488 nm air-cooled argon-ion laser while the fluorescence emission was measured through a 585/21 bandpass filter

All data were acquired in a logarithmic mode. Threshold settings were adjusted, so that the cell debris was excluded from the data acquisition. 10000 cells were measured for every sample. Data analysis was performed afterwards with FCSExpress V3 and MS Excel Software. The fluorescence emission of the immunofluorescence stained and surface-displayed antibody fragment 2F5Fab was normalized to the cell size, by calculation of ratio FL1/FSC. For each individual clone the geometric mean of FL1/FSC ratio of all 10000 measured cells was calculated and compared to the geometric mean of the FL1/FSC ration of a control strain not co-overexpressing a homologous gene. (P.pastoris 2F5Fab displaying strain transformed with an empty pLIB plasmid). Strains with an enhanced 2F5Fab cell surface signal were used for preparation of genomic DNA (DNeasy Blood and Tissue Kit, Qiagen 69504). Identification of co-overexpressed homologous genes was done by PCR amplification using pLIB specific oligonucleotide primer (GAPLibamp (SEQ ID No. 16) and Shble3out (SEQ ID No. 17), Table 6) and sequencing of the PCR product.

TABLE 6
Oligonucleotide primer for PCR amplification and
sequencing of co-overexpressed genes

| | |
|---|---|
| GAPLibamp | 5′-ACCACCAGAATCGAATATAAAAGGCGAACACCTTTC |
| | CCAATTTTGGTTT-3′ (SEQ ID No. 16) |
| Shble3out | 5′-GCGACCCGGCCGGCAACTGCGTGCACTTCGTGGCCG |
| | AGGAGCAGGACTGA-3′ (SEQ ID No. 17) |

Sequences were blasted against P.pastoris genomic sequence, S.cerevisiae genome database and NCBI database. From the output of the 5^{th} sorting round three open reading frames of P.pastoris could be identified.

RPPA12105 P. pastoris IG-66. no functional annota-
tion; preliminarily annotated as cLC52 (cDNA-
library clone 52)
Coding sequence (SEQ. ID No 1):
ATGAGTTTTCAAGTCTACAACATGGTGTCCTTGCCAGCACCGCAAGCAAC
ATCATTCTCCCAACATCCGAAACTTCCAACATTGGAAGTGATACAACGGC
CACCACTCACTGGAAGACATGGCATTCACGAAGAAGAGATCAAGCTACCT
TCCTTTAGATCTTTACTGGCTAGTGCGGGCACTGCCGCTGCTCTTGAATA
TCCCCCCAGGATTCAACCCCAATTTGCAACAGGGGCACCTGCACCTGAGC
AAGCATTTTATCAGCCCCTAAATACCGTGGGAGTCACGAGGCCCAACATT
CACACCTCCATCACTCCACCACTGGAGGCGCAGGACCTCCCTCACCAACA
AAAGTTTATGGAACCATTTGCGTCCTTCCCCAATACAGTAGTGTCTTCTA
CACCCAACACAGCCATCAGAAAATACAAGTGCAAGATCTGCGAGAGGTCT
TTCACAACGTCGGGCCACTTAGCTCGTCACACACGTATTCACACTGGTGA
AAAGAAGCATGAGTGTCCTTTCGAAGGCTGTTCTGCTAGGTTCAGCCGTC
AGGATAACTGCATGCAACATTACAAGACGCATGTTAATACCAAGTCGAAA
CGAAAGAGATCTAGATTGAGAAGTAAATAGGTA
RPPA10316 P. pastoris IG-66 Annotation: RPL33A by
homology to S. cerevisiae
Coding sequence (SEQ. ID No 2):
ATGGCTGAGGAAAACAGATTATACGTTAAGGGTAAGCACGTGTCTTTCCA
ACGTTCCAAGAGTGTTATCCACCCAAAGACTTCCTTGATCAAAATTGAGG
GTGTCGAAAACTCAAAGGATGCTGAGTTCTACATTGGTAAGAGAATCGCT
TACGTCTACAAAGGTGTAAAGGCTATCAACGGTACAAAGGTCAGAGTCAT
GTGGGGAACAGTCACCAGAACTCACGGAAACTCTGGTGTTGTCAGAGCTA
AGTTCGAACGTAACCTTCCAGGTCAGAGTTTTGGTTCCACTGTCAGAATC
ATGCTTTACCCATCTAACATCTAAATT
Genomic sequence including an intron (SEQ. ID No
3):
ATGGCTGAGGAAAACAGATGTATGTACTATTAAATCGCAAGGTGACTGGT
AAATGATGAAGATGATCGGCTTAATGAGAAGAATAATTGAAAACATAGCC
AGAAAGTTTTGATTTCTGTTAAACCAAGCTGAAATGATGGGTTGGGAATT
TTTTATTAAGCCAAATGAACATTCTTACAACTTTAACCAACGGATATTAT
ATATGGCTGAGCCAGCGTTATTCCAAAAGCACAAGTGGGAACAGGTCTCT
AAGTTTTTTCTTTACTTTCAAGTTGAGTTGACCGCTTCATCTTCATTTTC
TCCGACTATCACCACCTTCTCACTATATTTTTTAGATTCACGAGGTTACT
AACTTTGCTATAGTATACGTTAAGGGTAAGCACGTGTCTTTCCAACGTTC
CAAGAGTGTTATCCACCCAAAGACTTCCTTGATCAAAATTGAGGGTGTCG
AAAACTCAAAGGATGCTGAGTTCTACATTGGTAAGAGAATCGCTTACGTC
TACAAAGGTGTAAAGGCTATCAACGGTACAAAGGTCAGAGTCATGTGGGG
AACAGTCACCAGAACTCACGGAAACTCTGGTGTTGTCAGAGCTAAGTTCG
AACGTAACCTTCCAGGTCAGAGTTTTGGTTCCACTGTCAGAATCATGCTT
TACCCATCTAACATCTAAATT
RPPA09410 P. pastoris IG-66 no annotation (working
name cLC61 cDNA-library clone 61)
Coding sequence (SEQ. ID No 4):
ATGGCTCCCAGAACACTACCAGAAGACTTAATTCCCTCCCTATACGACTT
GCACATCTACAACTTCCAACCCGAAAAAAAGACTTATGATGGAGACATTG
TCATCCACTTGGAGGTGAAGGAGCCCACTGATGAAGTGGTCTTCAATGCC
AAGGATTTGGAATTGAAAGACGTACATGTCTTCCACAATGTCAACAAGTC
TGAAAACGAAATCCCCGTTAAGGAGATTGTTGATAACGAGCTCATCACAA
TTAAGCTCAAAGAGAAGGTTACTTCCGGAACGTTGCTGGTGAATATTTCC
TTCACCGGTAACATTCAATCTGATAAAATTGGATTTTACAAGGGAGACAC
AGATGTGGAAGGAAGAGTCACATACACTACAAACCTTACCACTCCAAATG
CCAGGTTGGCATTCCCATGTCTTGATAACATATTGTTGAAAGCTCCATTC
AAGTTCGGAGTAACTGCCAATCCAGGACAATTAGTGAGTTCCATTTTGGA
TCTAAGCTCTGAGGCTGACGTCTTGAATGACAATGACGATGTGATTGGTA
CGAGATACCAATACCAAGTGAGTGAGCCAATAGCCCCAGCTTTACTGGAG
TGGACCATTCATATTTAACGA

Identified open reading frames were amplified from pLIB cDNA-Library with specific oligonucleotide primers (Table 7) The P. pastoris Kozac sequence (GCC) was inserted directly before the start codon (ATG). The non template coded restrictions sites SbfI and SfiI were added by using corresponding forward and backward primer. After treatment with the restriction enzymes SbfI and SfiI the PCR products of the correct size were cloned into pPuzzle_ZeoR_Pgap_eGFP_AOXTT plasmid (Gasser et.al. 2008) replacing the open reading frame of eGFP (digested with SbfI and SfiI followed by treatment with alkaline phosphatase). The resulting plasmids (pPZ-RPL; pPZ-cLC52, pPZ-cLC61) and an empty pPuzzle_ZeoR_Pgap_AOXTT were used to transform P. pastoris SMD1168 displaying 2F5Fab after restriction digest with Ascl. After one round of replica plating, to individualize single clones, single colonies were used to inoculate pre-cultures on YPD media. After incubation at 28° C. at 180 rpm for about 20 hours these pre-cultures were used to inoculate an expression culture with OD600=0.1. Aliquots of expression cultures (grown to mid log phase at 28° C., 180 rpm) were used for immunfluorescence staining and flow cytometry analysis. Expression levels of different strains were compared by calculation of the geometric mean of FL1/FSC ratio (Table 8).

TABLE 7
Oligonucleotide primer for PCR amplification of
identified genes

| | |
|---|---|
| RPL33_ | 5′-GATTCACCTGCAGGCCATGGCTGAGGAAAACAGATTAT |
| #1_SbfI | ACGTTAAGGGT-3′ (SEQ ID No. 18) |
| RPL33_ | 5′-GGATGGCCGAGGCGGCCTTAGATGTTAGATGGGTAAAG |
| #2_SfiI | CATGATTCTG-3′ (SEQ ID No. 19) |
| cLC52_ | 5′-GATTCACCTGCAGGCCATGAGTTTTCAAGTCTACAACA |
| #1_SbfI | TGGTGTCCTTGCCAG-3′ (SEQ ID No. 20) |
| cLC52_ | 5′-GGATGGCCGAGGCGGCCCTATTTACTTCTCAATCTAGA |
| #2_SfiI | TCTCTTTCGTTTCGAC-3′ (SEQ ID No. 21) |
| cLC61_ | 5′-GATTCACCTGCAGGCCATGGCTCCCAGAACACTACCAG |
| #1_SbfI | AAGACTTAATTCCCTCC-3′ (SEQ ID No. 22) |
| cLC61_ | 5′-GGATGGCCGAGGCGGCCTTAAATATGAATGGTCCACTC |
| #2_SfiI | CAGTAAAGCTG-3′ (SEQ ID No. 23) |

TABLE 8
Expression levels

| Strain | Mean FL1/FSC ratio | fold change |
|---|---|---|
| Control | 0.2676 | 1 |
| RPL33A | 0.3205 | 1.20 |
| cLC52 | 0.4663 | 1.74 |
| cLC61 | 0.3889 | 1.45 |

### Example 2

### Co-Overexpression of Novel Helper Factor Genes in a Strain of P.pastoris Overexpressing Recombinant Human Trypsinogen (rhTRP)

a) Construction of Co-Overexpression Plasmids

To generate a plasmid suitable for co-overexpression of homologous gene of P.pastoris in a strain already expressing a heterologous protein from a vector confirming resistance to Zeocin (see Example 2 step b), in a first step the KanMX4 cassette (confirming Geneticin resistance in P. pastoris) was amplified by PCR from pFA6kanMX4 plasmid (Longtine et.al. 1998). Restriction sites for Kpnl were added by using specific oligonucleotide primer KanR #1 (SEQ ID No. 24) and KanR#2 (SEQ ID No. 25) (Table 9). The Kpnl treated PCR fragment was cloned instead of the Zeocin resistance cassette between both Kpnl sites of pPuzzle_ZeoR_Pgap_eGFP_AOXTT (Gasser et.al. 2008). The resulting vector was called pPkanR.

TABLE 9

| | |
|---|---|
| KanR#1 | 5′-CCGAGGTACCGACATGGAGGCCCAGAATA-3′ |
| | (SEQ ID No. 24) |
| KanR#2 | 5′-CCGAGGTACCAGTATAGCGACCAGCATTCA-3′ |
| | (SEQ ID No. 25) |

All genes of interest for co-overexpression (see Example 1) were amplified via PCR from P. pastoris cDNA library. Non template coded P. pastoris Kozak sequence and restriction sites for SbfI and SfiI were added by using respective forward and backward oligonucleotide primer (Table 7). Sbfi and Sfil treated PCR products were cloned into pPKanR (SbfI and SfiI digested and treated with alkaline phosphatase). Novel co-overexpression plasmids pPKanR-RPL, pPKanR-cLC52 and pPKanR-cLC61 were transformed into E.coli Top10 (Invitrogen). Restriction endonuclease digest and sequencing was done to verify the correct identity of the constructed plasmid.

b) Construction of P. pastoris Strains Co-Overexpressing Recombinant Human Trypsinogen and a Novel Helper Factor Gene

The plasmids pPKanR-RPL, pPKanR-cLC52 and pPKanR-cLC61, obtained from cloning procedure described in Example 2 step a) were used to transform a strain of P. pastoris pre-selected for the high level expression of recombinant human trypsinogen under the control of the GAP promoter (Hohenblum et.at 2004). Selection was based on Zeocin resistance for the trypsinogen gene and Geneticin resistance for the helper factor genes.

To evaluate the effect of co-overexpressed helper factor gene the trypsinogen expressing strain was also transformed with a pPkanR plasmid without helper factor gene.

c) Culturing Transformed P. pastoris Strains in Shake Flask Cultures

5 ml YP-medium (10 g/l yeast extract, 20 g/l peptone) containing 10 g/l glycerol were inoculated with a single colony of P. pastoris strains from step a) Example 3 and grown overnight at 28° C. Aliquots of these cultures (corresponding to a final OD600 of 0.1) were transferred to 12.5 ml of expressions culture medium (per liter: 10 g yeast extract, 10 g pea-peptone, 10.2 g (NH4)2PO4, 1.24 g KCl, 0.1 g CaCl2, pH 5.0 adjusted with HCl) supplemented with 20 g/L glucose and incubated for 60 h at 28° C. C at vigorous shaking in 100 ml Erlenmeyer flasks. The same amounts of substrate were added repeatedly 4 times every 12 h, before cells were harvested by centrifugation at 2500×g for 10 min at room temperature and prepared for analysis (biomass determination by measuring the cell weight of 1 ml cell suspension), enzyme activity assay for trypsinogen quantification in the culture supernatant).

d) Evaluation of the Effect of Co-Overexpression of Novel Helper Factors by Quantification of rhTRP

Desalting of P. pastoris culture supernatant was done using small pre-packed size exclusions chromatography columns (Disposable PD-10 Desalting Columns 17-0851-01; GE Healthcare) 2.5 ml of supernatant were applied to the column and eluted with 3.5 ml of elution buffer (1 mM HCl). After elution 70 μl of 2M CaCl2 solution was added. To convert inactive trypsinogen to the active trypsin 300 μl of the desalted supernatant (+CaCl2) were mixed with 690 μl of activation buffer (50 mM TRIS/HCl pH 8.6; 40 mM CaCl2 0,15 g/L Enterokinase from porcine intestine Sigma-Aldrich E0632) and incubated for two hours at 37° C. 165 μl of the activation mixture was mixed with 1000 μl of N_{α}-p-Tosyl-L-arginine-methyl-ester-hydrochloride (TAME) solution, containing 446 mg/L TAME (Sigma-Aldtrich T4626) dissolved in dilution buffer (50 mM TRIS/HCl pH 8.1; 40 mM CaCl2) and an absorption kinetic at 247 nm was measured in a spectrophotometer over a time period of 5 min. If necessary, activated trypsin solution was diluted with dilution buffer to hit the linear range (ΔA₂₄₇/min<0.3) of this method. A trypsin concentration of 1 g/L corresponds to ΔA₂₄₇/min=0.101.

The evaluation of the data showed that the co-overexpression of PpcLC52, PpRPL33A and PpcLC61 had a significant positive effect on the amount of secreted recombinant human trypsinogen (Table 10).

### Example 3

### Co-Overexpression of the Novel Helper Factor PpcLC52 in a P.pastoris Strain Expressing Fab Fragment of the Anti-HIV1 Antibody 2F5

a) Construction of the P. pastoris Strain SMD 1168 Secreting the Fab Fragment of the Monoclonal Anti-HIV1 Antibody 2F5.

Plasmids combining the expression cassettes for both Fab chains on one vector were produced by double digestion of the light chain vector with BgIII and BamHI, and subsequent insertion into the unique BamHI of the vector pGAPZalfaA already containing a single copy of the expression cassette of the heavy chain fragment. Plasmids were then linearized with Avrll prior to electrotransformation into P. pastoris.

All constructed expression cassettes were checked by DNA sequencing with the GAP forw/AOX3′ backw (Invitrogen)

The resulting plasmid was used to transform P. pastoris SMD1168 (Invitrogen) after restriction digest with NdeI. Transformants were selected for Zeocin resistance and screened for high level expression of the Fab fragment of the monoclonal anti-HIV1 antibody 2F5.

b) Construction of a P. pastoris Strain Co-Overexpressing 2F5 Fab and the Helper Factor PpcLC52:

Transformation of a P. pastoris strain obtained in step a) Example 3 was carried out with the plasmid pKanR-cLC52 of step a) Example 2, which was linearized with Ascl. The plasmid was introduced into the cells by electrotransformation. The transformed cells were cultivated on YP-agar containing 20 g/L glucose and 500 mg/L Geneticin. As a control, the 2F5 Fab expressing strain was also transformed with a pPkanR plasmid without helper factor gene.

c) Culturing Transformed P. pastoris Strains in Shake Flask Cultures

5 ml YP-medium (10 g/l yeast extract, 20 g/l peptone) containing 10 g/l glycerol were inoculated with a single colony of P. pastoris transformants from b) Example 3 and grown overnight at 28° C. Aliquots of these cultures (corresponding to a final OD600 =0.1) were transferred to 10 ml of expressions culture medium (per liter: 10 g yeast extract, 10 g peptone, 100 mM potassium phosphate buffer pH 6.0, 13.4 g yeast nitrogen base with ammonium sulfate, 0.4 mg biotin) supplemented with 20 g/l glucose and incubated for 48 h at 28° C. C at vigorous shaking in 100 ml Erlenmeyer flasks. The same amounts of substrate were added repeatedly 3 times every 12 h, before cells were harvested by centrifugation at 2500×g for 5 min at room temperature and prepared for analysis (biomass determination by measuring the cell weight of 1 ml cell suspension, ELISA for Fab quantification in the culture supernatant).

d) Evaluation of the Effect of Co-Expression Helper Factors by Quantification of 2F5Fab

To determine the amount of secreted recombinant expressed 2F5 Fab, 96 well microtiter plates (MaxiSorb, Nunc, Denmark) were coated with anti-human IgG (Fab specific) overnight at RT (Sigma-Aldrich 15260; 1:1000 in PBS, pH 7.4), before serially diluted supernatants of P. pastoris cultures secreting 2F5 Fab from step d) (starting with a 1:50 dilution in PBS/Tween20 (0.1%)+2% BSA) were applied and incubated for 2 h at RT. A human Fab fragment of IgG (Rockland) was used as a standard protein at a starting concentration of 200 ng/ml. After each incubation step the plates were washed three times with PBS containing 0.1% Tween 20 (adjusted to pH 7.4). 100 μl of anti-human-kappa light chain—AP conjugate as secondary antibody (Sigma-Aldrich A-3813 1:1000 in PBS/Tween+2% BSA) were added to each well, and incubated for 1 h at RT. After washing, plates were stained with pNPP, 1 mg/ml p-nitrophenyl phosphate in detection buffer, (0.1 N Na₂CO₃/NaHCO₃ pH 9.6) and read at 405 nm (reference wavelength 620 nm). The data are summarized in Table 10.

### Example 4

a) Construction of a P.pastoris Strain Overexpressing Recombinant Porcine Trypsinogen.

For expression of recombinant porcine trypsinogen (rpTRP) a codon-optimized artificial gene was synthesized (Geneart Germany) During DNA synthesis a P. pastoris Kozak sequence (GCC just before the start codon ATG), the S. cerevisiae mating factor alfa secretion leader sequence, and sites for the restriction enzymes SbfI and SfiI, flanking the open reading frame were added. The coding sequence of the rpTRP gene (flanked by the Kozak sequence) was cut out of the delivered plasmid using SbfI and SfiI, and cloned into a SbfI, SfiI and alkaline phophatase treated plasmid pPuzzle_ZeoR_Pgap_eGFP_AOXTT (Gasser et.at 2008) replacing the open reading frame of the eGFP gene. The ligated plasmid was transformed into E.coli TOP10 (Invitrogen) and plated on Zeocin containing LB-agar. Restriction endonuclease analysis was performed to confirm the correct identity of the plasmid pPZ_Pgap_rpTRP_AOXTT. After linearization with Ascl the vector was used to transform the P.pastoris strain X33 (Invitrogen) and plated on Zeocin and glucose containing YP-agar.

b) Construction of P. pastoris Strains Co-Overexpression rpTRP and the Novel Helper Factor Gene PpcLC52

The plasmid pPKanR-cLC52 obtained from cloning procedure described in Example 2a) were used to transform a strain of P.pastoris pre-selected for the high level expression of rpTRP under the control of the GAP promoter, as described in Example 4a). Selection was based on Zeocin resistance for the rpTRP gene and Geneticin resistance for the helper factor gene. To evaluate the effect of co-overexpressed helper factor genes on the expression of rpTRP, the trypsinogen expressing strain was also transformed with a pPkanR plasmid without helper factor gene.

c) Culturing Transformed P. pastoris Strains in Shake Flask Cultures

Cultivations of P. pastoris strains obtained from Example 3 step b) were done as described in Example 2 step c) with little modification. Instead of measuring the wet cell weight, the yeast dry mass of 10 ml cell suspension was appointed to determine the produced amount of biomass.

d) Evaluation of the Effect of Co-Overexpression of the Helper Factor by Quantification of rhTRP

Determination of the amount of recombinant porcine trypsinogen was performed as described in Example 3 step d for recombinant human trypsinogen). The data are shown in Table 10.

TABLE 10

| | coexpressed | Product | fold change | fold change |
|---|---|---|---|---|
| product | gene | concentration | product | product/biomass |
| rhTRP | RPL33A | 5.08 | 1.24 | 1.26 |
| rhTRP | cLC52 | 7.49 | 1.84 | 1.80 |
| rhTRP | cLC61 | 4.66 | 1.14 | 1.19 |
| rhTRP | control | 4.08 | — | — |
| 2F5 | cLC52 | 6.65 | 1.26 | 1.83 |
| 2F5 | control | 5.27 | — | — |
| rpTrp | cLC52 | 37.91 | 1.41 | 1.34 |
| rpTrp | control | 26.87 | — | — |

### Example 5

### Cultivation of a P. pastoris Strain Co-Overexpressing Porcine Trypsinogen and PpcLC52 in Fermentation Cultures

Batch-Fermentation of P. pastoris

Fermentations were performed in a bioreactor, which is a pilot scale for industrial production, with a total volume of 5.0 l (Minifors, Infors, Switzerland) with a computer-based process control system. The media were as follows:

All chemicals for PTM₁ trace salts stock solution were from Riedel-de Haën, except for biotin (Sigma), and H₂SO₄ (Merck Eurolab).

PTM1 trace salts stock solution contained per litre: 6.0 g CuSO₄.5H₂O, 0.08 g NaI, 3.0 g MnSO₄. H₂O, 0.2 g Na₂MoO₄.2H₂O, 0.02 g H₃BO₃, 0.5 g CoCl₂, 20.0 g ZnCl₂, 65.0 g FeSO₄.7H₂O, 0.2 g biotin and 5.0 ml H₂SO₄ (95%-98%).

Batch medium contained per litre: 2.0 g/L Citric-acid xH₂O, 12.6 g (NH₄)₂HPO₄, 0.022 g CaCl₂ x2H₂O, 0.9 g KCl, 0.5 g MgSO₄ x7H₂O, 40 g glycerol, 4.6 ml PTM1 trace salts stock solution and 0.4 mg D-Biotin. The pH was adjusted to 5.0 with 25% HCl.

The dissolved-oxygen concentration was maintained above 20% saturation by controlling the stirrer speed between 600 and 1250 rpm. Aeration rate was 120 L* h⁻¹ air. The temperature was 25° C., and the pH was controlled with NH₃ (25%). Before starting the fermentation, the pH of the batch medium was set to 5.0.

The batch phase of approximately 24 to 30 h led to a dry biomass concentration of approximately 20 g l⁻¹. In the feed media glucose (composition depending on feed strategy, see step b), step c) and step d)) serves as a carbon source. The pH was 5.0 during batch, and kept at 5.0 throughout the fermentation.

Samples were taken at the end of the glycerol batch phase, during and/or at the end of the feeding phase. 10m1 of cell suspension were centrifuged to separate the cells from culture supernatant, then the cell pellets were washed with water and dried at 105° C. for 24 h to determine cell dry weight (YDM). The supernatant was frozen at −20° C. and keep until trypsinogen assay (as described for human trypsinogen step c example 3).

a) Strains

A strain obtained from Example 4 step b),co-overexpressing recombinant porcine trypsinogen and PpcLC52, and the “wildtyp” strain were used to inoculate a preculture on YPG (10 g/L yeast extract, 20 g/L peptone, 10 g/L glycerol). Precultures were incubated with shaking at 28° C. for 24 h. To remove complex media components cells were harvested by centrifugation at 1500×g for 10 min and re-suspended in batch media. Resulting cells suspension was used to inoculate the initial batch phase at a starting optical density at 600 nm of 1.0

b) Chemostat-Fermentation of a P. pastoris Strain Co-Overexpressing rpTRP and PpcLC52.

After an initial batch phase (batch media volume 1.75 litre) the fermentation was run in a chemostat mode with a specific growth rate of μ=0.1 and a biomass concentration (yeast dry mass) of 25 g/L. Feed-media flow (1.0 g/L Citric-acid xH₂O; 4.4 g/L (NH₄)₂HPO₄, 0.01 g/L CaCl₂ x2H₂O, 1.7 g/L KCl, 0.7 g/L MgSO₄ x7H₂O, 55 g/L glucose xH₂O, 1.6 ml/L PTM1 trace salts stock solution and 0.4 mg/L D-biotin; pH was adjusted to 5.0 with 25% HCl) and cell suspension harvest flow were set to 175 g/h. Air flow was keep constant a 120 L/h. Samples were taken at the end of the batch phase and after 5 volume changes (50 h feeding).

On glucose as single carbon source and steady-state condition with a specific growth rate of 0.1 h⁻¹ the product concentration and the specific productivity of the PpcLC52 co-overexpressing strain was 1.4 fold higher compared to the control strain.

c) Fedbatch-Fermentation of a P. pastoris Strain Co-Overexpressing rpTRP and PpcLC52 with Linear Feeding.

After an initial batch phase (batch media volume 1.6 litre) the fermentation was run in a Fedbatch mode with a linear feeding profile. Feed-media flow (0.28 g/L CaCl₂ x2H₂O, 8.4 g/L KCl 5.2 g MgSO₄ x7H₂O, 464 g/L glucose x H2O, 10.0 ml/L PTM1 trace salts stock solution and 0.4 mg/L D-Biotin) was set to 17.5 g/h. Air flow was keep constant a 120 L/h. Samples were taken after the batch phase, every 24 h during the feeding period and at the end of feeding (100 h).

The comparison of PpcLC52 co-overexpressing strain (rpTRP_cLC52) and the non co-overexpression strain (rpTRP_wt) showed an equal (linear) accumulation of biomass. The average specific productivity of the rpTRP_cLC52 strain was 1.3 fold higher, and the total product amount accumulated with rpTRP_cLC52 was 1.2 higher compared to rpTRP_control.

c) Fed-Batch Fermentation of a P.pastoris Strain Co-Overexpressing Porcine Trypsinogen and PpcLC52 with Constant Specific Growth Rate of p=0.1 h⁻¹

After an initial batch phase (batch media volume 1.4 litres) the fermentation was run in a fed-batch mode with exponential feeding profile. Feed-media flow (0.28 g/L CaCl₂ x2H₂O, 8.4 g/L KCl 5.2 g MgSO₄ x7H₂O, 464 g/L glucose xH₂O , 10.0 ml/L PTM1 trace salts stock solution and 0.4 mg/L D-biotin) was adjusted by a PID controller to keep a constant specific growth rate of μ=0.1 h⁻¹. The dissolved-oxygen concentration was maintained at 20% saturation by controlling the stirrer speed between 600 and 1250 rpm and the air flow between 120 and 500 L*h⁻¹. Due to limitation in oxygen transfer rate of the reactor the process ran in oxygen limitation after 16 h of feeding. Samples were taken after the batch phase, 6 times during the feeding period and at the end of feeding (approximately. 25 h)

The comparison of PpcLC52 co-overexpressing strain (rpTRP_cLC52) and the non co-overexpression strain (rpTRP_wt) showed an equal accumulation of biomass. The total product amount (cell free culture volume * product concentration) of the PpcLC52 co-overexpressing strain was 1.2 fold higher at the end of the fed batch phase compared to the control strain. The specific productivity of the rpTRP_cLC52 strain was up to 2.2 fold higher.

### Example 6

### Construction of Wildcard Strains

The plasmids pPKanR-RPL, pPKanR-cLC52 and pPKanR-cLC61, obtained from cloning procedure described in Example 2 step a) are used to transform a strain of P. pastoris which does not express a POI. Selection is based on Geneticin resistance for the helper factor genes. These transformed strains can further be used as improved production strains for POIs.

References

Archer, D., Jeenes, D. and Mackenzie, D. (1994). Strategies for improving heterologous protein production from filamentous fungi. Antonie Van Leeuwenhoek 65, 245-50.

Gething, M. and Sambrook, J. (1992). Protein folding in the cell. Nature 355, 33-45.

Hohenblum, H., et al. (2003) Assessing viability and cell-associated product of recombinant protein producing Pichia pastoris with flow cytometry. J Biotechnol 102, 281-290

Kurtzman C P. (2005). Description of Komagataella phaffii sp. nov. and the transfer of Pichia pseudopastoris to the methylotrophic yeast genus Komagataella. Int J Syst Evol Microbiol 55, 973-976.

Lang, C. and Looman, A. (1995). Efficient expression and secretion of Aspergillus niger RH5344 polygalacturonase in Saccharomyces cerevisiae. Appl Microbiol Biotechnol 44, 147-56.

Macauley-Patrick, S., Fazenda, M. L., McNeil, B. and Harvey, L. M. (2005). Heterologous protein production using the Pichia pastoris expression system. Yeast 22, 249-70.

Mattanovich, D., Gasser, B., Hohenblum, H. and Sauer, M. (2004). Stress in recombinant protein producing yeasts. J Biotechnol 113, 121-35.

Mori, K., Ogawa, N., Kawahara, T., Yanagi, H. and Yura, T. (2000). mRNA splicing-mediated C-terminal replacement of transcription factor Hac1p is required for efficient activation of the unfolded protein response. Proc Natl Acad Sci USA 97, 4660-5.

Porro, D., Sauer, M., Branduardi, P. and Mattanovich, D. (2005). Recombinant protein production in yeasts. Mol Biotechnol 31, 245-59.

Punt, P. J., van Biezen, N., Conesa, A., Albers, A., Mangnus, J. and van den Hondel, C. (2002). Filamentous fungi as cell factories for heterologous protein production. Trends Biotechnol 20, 200-6.

Sauer, M., Branduardi, P., Gasser, B., Valli, M., Maurer, M., Porro, D. and Mattanovich, D. (2004). Differential gene expression in recombinant Pichia pastoris analysed by heterologous DNA microarray hybridisation. Microb Cell Fact 3, 17.

Shuster, J. (1991). Gene expression in yeast: protein secretion. Curr Opin Biotechnol 2, 685-90.

Stryer, L. (1995). Biochemie. Spektrum der Wissenschaft Verlags GmbH.

LONGTINE, M. S., A. MCKENZIE, 3RD, D. J. DEMARINI, N. G. SHAH, A. WACH et al., 1998 Additional modules for versatile and economical PCR-based gene deletion and modification in Saccharomyces cerevisiae. Yeast 14: 953-961 Hohenblum H, Gasser B, Maurer M, Borth N, Mattanovich D. Effects of Gene Dosage, Promoters, and Substrates on Unfolded Protein Stress of Recombinant Pichia pastoris Biotechnol Bioeng. 2004 Feb. 20;85(4):367-75.

Gasser, B., Maurer, M., Gach, J., Kunert, R. and Mattanovich, D. (2006). Engineering of Pichia pastoris for improved production of antibody fragments. Biotechnol Bioeng 94, 353-61.

Gasser, B., Mattanovich, D., Sauer, M., Stadlmayr, G. (2008) WO 2008/128701A2 Expression System

1. A eukaryotic host cell comprising a recombinant nucleotide sequence encoding an expression enhancer, wherein the expression enhancer is selected from the group consisting of cLC52, RPL33 and cLC61.

2. The host cell of claim 1, wherein said nucleotide sequence is from P. pastoris.

3. The host cell of claim 1, wherein the host cell is selected from the group consisting of a fungal cell, a mammalian cell, and a plant cell.

4. The host cell of claim 3, wherein the host cell is a yeast cell of the Pichia genus.

5. The host cell of claim 1, wherein the host cell is a wildcard host cell for the introduction of a gene of interest encoding a protein of interest (POI).

6. The host cell of claim 1, wherein the host cell is a producer cell comprising a recombinant nucleotide sequence encoding a protein of interest (POI), and is capable of producing the POI.

7. The host cell of claim 1, wherein the host cell is a P. pastoris cell and comprises
a gene of interest encoding a protein of interest.

8. (canceled)

9. A method of producing a host cell comprising:
(a) providing a wildcard host cell comprising a recombinant nucleotide sequence encoding an expression enhancer, wherein the expression enhancer is selected from the group consisting of cLC52, RPL33 and cLC61, and
(b) introducing a nucleotide sequence encoding a protein of interest into the host cell.

10. A method of producing a host cell comprising:
(a) providing a eukaryotic host cell,
(b) introducing a nucleotide sequence encoding at least one of cLC52, RPL33 and cLC61 into the host cell, and
(c) introducing a nucleotide sequence encoding a protein of interest into the host cell.

11. A method of producing a protein of interest (POI) in a eukaryotic cell, comprising:
(a) providing a producer host cell comprising:
a recombinant nucleotide sequence encoding an expression enhancer, wherein the expression enhancer is selected from the group consisting of cLC52, RPL33 and cLC61; and
a recombinant nucleotide sequence encoding a protein of interest (POI),
(b) co-expressing the POI and the expression enhancer in a cell culture, and
(c) obtaining the POI from the cell culture.

12. A method according to claim 11, wherein said POI is a polypeptide selected from the group consisting of a serum protein, an enzyme, a hormone, a signalling molecule, a matrix protein, and a fragment or derivative thereof.

13. A method according to claim 11, wherein said POI mediates the production of a host cell metabolite.

14. A method according to claim 11, wherein the recombinant nucleotide sequence encoding a POI is contained in a plasmid suitable for integration into the genome of the host cell or for autonomous replication in the host cell.

15. A method according to claim 13, wherein the plasmid is a eukaryotic expression vector comprising a secretion leader sequence effective to cause secretion of the POI from the host cell and/or a promoter sequence effective to cause expression of the POI in the host cell.

16. A plasmid comprising:
(a) a recombinant nucleotide sequence encoding a protein of interest, and
(b) a nucleotide sequence encoding a protein selected from the group consisting of cLC52, RPL33, and cLC61.

17. The host cell of claim 3, wherein the host cell is a yeast cell.

18. The host cell of claim 4, wherein the host cell is a P. pastoris cell.

19. A method according to claim 12, wherein the serum protein is an immunoglobulin or serum albumin.

20. A method according to claim 15, wherein the expression vector is a yeast expression vector.
